(19) United States
(12) Patent Application Publication
(10) Pub. No.: US 2023/0218796 A1
(43) Pub. Date: Jul. 13, 2023

(54) Tissue adhesive for use in a treatment method in which an ophthalmological implant is implanted in a human or animal patient, and ophthalmological implantation system

(71) Applicant: Carl Zeiss Meditec AG, Jena (DE)

(72) Inventor: André Wolfstein, Berlin (DE)

(21) Appl. No.: 18/185,191

(22) Filed: Mar. 16, 2023

Related U.S. Application Data
(63) Continuation of application No. PCT/EP2021/074960, filed on Sep. 10, 2021.

(30) Foreign Application Priority Data
Sep. 18, 2020 (DE) 10 2020 124 372.3

(51) Int. Cl. A61L 24/00; A61K 31/513; A61K 31/365; A61K 31/337; A61K 31/78; A61K 38/18; A61L 24/04

(52) U.S. Cl. CPC A61L 24/0015; A61K 31/513; A61K 31/365; A61K 31/337; A61K 31/78; A61K 38/1841; A61L 24/0031; A61L 24/043; A61L 2430/16; A61L 2300/412; A61L 2300/414; A61L 2300/216; A61L 2300/416

(57) ABSTRACT

The disclosure relates to a tissue adhesive for use in a treatment method in which an ophthalmological implant is implanted in a human or animal patient and the ophthalmological implant is connected, at least partially in an integrally bonded manner, to eye tissue of the patient via the tissue adhesive. The disclosure also relates to an ophthalmological implantation system including an ophthalmological implant for implantation in a human or animal eye and to a tissue adhesive via which the ophthalmological implant is connectable, at least partially in an integrally bonded manner, to eye tissue of the patient.

### CROSS REFERENCE TO RELATED APPLICATIONS

This application is a continuation application of international patent application PCT/EP2021/074960, filed Sep. 10, 2021, designating the United States and claiming priority from German application 10 2020 124 372.3, filed Sep. 18, 2020, and the entire content of both applications is incorporated herein by reference.

### TECHNICAL FIELD

The disclosure relates to a tissue adhesive for use in a method of treatment in which an ophthalmological implant is implanted in a human or animal patient, and to an ophthalmological implantation system for such a method of treatment.

In the case of use of ophthalmological implants, for example intraocular lenses (IOLs) or artificial capsular bags, interactions between the implant and adjacent biological tissue mean that various complications can occur. For example, so-called posterior capsule opacification (PCO, cataracta secundaria) occurs in some cases after cataract operations. PCO is post-operative clouding of the lens capsule after surgical extraction of a natural lens. The remaining lens epithelial cells (E cells) in the equatorial region of the capsular bag are mitotically active and can transform into fibroblasts. These then trigger a kind of wound healing, involving formation of collagen-containing connective tissue. Since some fibroblast subtypes not only migrate onto the inner side of the capsular bag, but can also contract, wrinkles form in the capsular bag. The clouding of the capsule is therefore the result of a wound healing process and associated scarring. Since the lens clouding caused thereby has causes other than the original cataract disease, this is referred to as an “aftercataract” or a “secondary cataract”. For those affected, a clinically significant aftercataract can lead to a reduction in visual acuity, in color perception and in contrast vision and to increased glare.

Secondary cataract is a common complication after extracapsular cataract extraction (ECCE) and the subsequent implantation of an intraocular lens (IOL) in the capsular bag. Without the implantation of an IOL in the empty capsular bag, the risk of an aftercataract is even more considerably increased, since unhindered cell migration to the posterior surface of the capsular bag is possible in this case. The implantation of an artificial capsular bag also harbors a comparatively high risk of an aftercataract or other complications that are caused by uncontrolled cell growth.

The incidence of a secondary cataract increases over time after a surgical procedure. A meta-analysis of the cases of cataracta secundaria for all existing types of IOLs showed an approximate average increase of 12% one year after surgery and an approximate average increase of 30% five years after surgery. The age of the affected patient also appears to be a crucial factor here, and so it has to be expected that almost all treated children and adolescents might suffer from a post-operative secondary cataract after a certain period of time.

US 2011/287067 A1 discloses the synthesis of reinforced adhesive complexes and the use thereof. The reinforced adhesive complexes consist of at least one polycation, at least one polyanion and a reinforcing component. The adhesive complexes described can then be cured in order to produce strong cohesive adhesives.

US 2019/070338 A1 discloses neurosupportive materials that have strong tissue adhesion and have been synthesized by crosslinking two polymers: gelatin-methacryloyl (GeIMA) and methacryloyl-substituted tropoelastin (MeTro). The materials developed have mechanical properties that are adjustable by variation of the GeIMA/MeTro ratio.

Yi Hong et al.: “A strongly adhesive hemostatic hydrogel for the repair of arterial and heart bleeds” (Nature Communications, vol. 10, no. 1, 14. 2019-05-14, XP055707391) discloses a photoreactive adhesive that simulates the composition of the extracellular matrix (ECM). This matrix hydrogel based on biomacromolecules is capable of rapid gelation and fixation in order to bond and to seal bleeding arteries and cardiac walls after irradiation with UV light.

U.S. Pat. No. 6 702 853 B1 discloses a system and a method of removing cataract cells in a lens capsule of an eye, and of inserting an intraocular lens into the lens capsule, wherein an adhesive has been applied to at least one of the surfaces thereof. Preferably, the adhesive is applied to the surface of the intraocular lens facing the cornea of the eye.

WO 2006/067638 A2 discloses amphiphilic block copolymers including at least one block containing hydrophilic units and at least one block containing hydrophobic units, wherein at least one hydrophobic block contains siloxane units. The block copolymers may especially be useful as tissue adhesive or as coating for an intraocular lens (IOL). As IOL coating, the block copolymers may be used, for example, to promote tissue adhesion for prevention of posterior capsular opacification.

It has not been possible to date to quantitatively remove all epithelial cells during eye operations without using toxic medicaments which can damage the endothelial cell layer of the cornea or other eye tissue. The vision problems caused by fibrosis and PCO are partially treated by so-called focal Nd: YAG capsulotomy. Nevertheless, fibrotic striae in particular can still impair the function of implants, for example an accommodatable IOL.

Until now, therefore, it has not been possible to achieve significant and prolonged prophylactic action against Cataracta secundaria and other medical complications that occur in association with the implantation of ophthalmological implants.

### SUMMARY

It is an object of the present disclosure to reduce the risk of PCO and fibrosis for methods of treatment in which an ophthalmological implant is implanted in a human or animal patient. It is a further object of the disclosure to provide an ophthalmological implantation system for such a method of treatment that can reduce the risk of PCO and fibrosis.

The objects are achieved by a tissue adhesive in accordance with the disclosure for use in a method of treatment in which an ophthalmological implant is implanted in a human or animal patient. The objects can further be achieved by an ophthalmological implantation system as disclosed herein.

A first aspect of the disclosure relates to a tissue adhesive for use in a treatment method in which an ophthalmological implant is implanted in a human or animal patient and the ophthalmological implant is at least partly cohesively bonded to tissue of the patient's eye via the tissue adhesive, wherein the tissue adhesive in the uncured state takes the form of an interpenetrating network and/or of a semi-interpenetrating network. In other words, the disclosure provides a tissue adhesive which, during a method of treatment in which, for example, a patient's eye lens is replaced by an ophthalmological implant, can be used to partly or fully cohesively bond the ophthalmological implant to the patient's capsular bag. Correspondingly, the tissue adhesive can be used for cohesive bonding of an artificial capsular bag to the patient's eye tissue and for further types of eye implantation. A tissue adhesive in the context of the present disclosure is understood to mean a pharmacologically tolerable adhesive designed to form a cohesive bond in vivo between the biological eye tissue and the ophthalmological implant adjoining the eye tissue in question. The word “cohesive” in the context of the present disclosure is understood to mean bonds where the adherends are held together by atomic or molecular binding forces, wherein the binding forces can include strong bonds and/or weak bonds (van der Waals interactions). The binding forces preferably include strong bonds, especially covalent bonds. A tissue adhesive in the context of the present disclosure is thus not a straight adhesion promoter such as fibronectin, vitronectin, laminin or glycoproteins, for example, but creates a cohesive bond between the eye tissue in question and the implant under chemical curing. Tissue adhesives are known per se for different surgical methods of wound closure without surgical suture material, but have not been described to date for the medical indication of the disclosure in the context of a generic eye operation. With the aid of the tissue adhesive of the disclosure, it is possible to immobilize and firmly bond the ophthalmological implant in the eye without damaging the eye tissue. It is thus possible to reliably prevent problems that commonly occur in generic methods of treatment, such as misalignment, tilting, rotation or detachment of the implant. Furthermore, such a capsular bag-based implantation can to date be associated with posterior capsule opacification (PCO) and fibrosis, which can especially be caused by residual lens epithelial cells. The possibility of cohesive bonding of the implant to the eye tissue with the aid of the tissue adhesive can, by contrast, reliably prevent undermining of the implant with lens epithelial cells and the like. Any lens epithelial cells and other cell types that remain in the eye in the operation can thus no longer cause PCO and fibrosis or similar complications that can impair the sight and functionality of an implant, for example an intraocular lens (IOL), especially an accommodating IOL, or of an artificial capsular bag. Because the tissue adhesive in the uncured state takes the form of an interpenetrating network and/or of a semi-interpenetrating network, it is advantageously possible to use network components that may be chemically identical or different. In addition, it is possible in this way to use two mutually immiscible polymer types and bond them to one another. Furthermore, monomers that polymerize by entirely different, non-interfering mechanisms may be subjected to simultaneous interpenetrating crosslinking. In this way, it is possible to optimize the mode and initiation of curing to the respective end use.

In a configuration of the disclosure, the tissue adhesive is provided as a composition with an active ingredient release system designed, in the implanted state of the tissue adhesive, to release at least one active pharmacological ingredient, preferably in a controlled manner, and/or the tissue adhesive is provided as a composition with an active pharmacological ingredient immobilized on the tissue adhesive. In this way, it is advantageously possible, after implantation, to use the tissue adhesive for local release of one or more pharmacologically active substances or for long-term positioning of the active ingredient at a desired site. In this way, it is possible to release one or more active ingredients—preferably in a controlled manner—specifically into the region in which they are required. Systemic or non-specific administration of the active ingredient and associated side effects can advantageously be avoided. For example, the active ingredient thapsigargin, in the case of free release into the aqueous humor in the anterior chamber, can damage the epithelial cell layer of the cornea. By contrast, preferably controlled release into the directly adjacent tissue ensures restriction of the pharmacological action to the site where it is required. In addition, the amount of the active ingredient can be reduced to a therapeutically necessary amount, which means that, in spite of improved and long-lasting efficacy, lowering of the risks of complication and considerable cost savings are achievable.

Further benefits arise in that the at least one active ingredient is designed to promote and/or inhibit at least one aspect from a group including proliferation, migration and differentiation of cells that occur in the human or animal eye. In other words, the active ingredient is designed to control the genesis of PCO or fibrosis by specifically promoting and/or inhibiting the proliferation, migration and/or differentiation of cells, for example lens epithelial cells in the implanted state. The inhibition of transformation, cell growth and/or cell division of such cells likewise advantageously prevents undermining or overgrowth of the implant “fixed” in the eye tissue via the tissue adhesive, which can particularly effectively suppress and prevent the genesis of PCO or fibrosis and related complications. Alternatively or additionally, it may be the case that the proliferation, migration and/or differentiation of cells, for example lens epithelial cells, is specifically promoted. This results in improved tissue growth through active cell cultivation and hence promotes natural wound healing. The proliferation and migration of the cells that occur in the human or animal eye can thus be controlled, such that these cells can no longer cause the aforementioned problems that lead to PCO for example. Furthermore, the induction and possible control of rapid fibrosis is advantageous since it increases the stability of the implant, rapidly ensures the ultimate positioning of the implant in the eye tissue, and shortens the healing time, for example after the cataract operation or after the insertion of an artificial capsular bag. The cells may in principle also occur in various cell forms, for example as fibroblasts, and/or as cell accumulations, for example as Wedl cells.

In a further configuration, the at least one active ingredient is selected from a group including 5-fluorouracil, thapsigargin, paclitaxel, growth factors, especially TGFβ, and angiogenesis inhibitors, and also derivatives, especially (meth)acrylate-modified derivatives, isomers and any mixtures thereof. 5-Fluorouracil is a cytostatic and, being a pyrimidine analog, belongs to the group of anti-metabolites. It has structural similarity to the pyrimidine base uracil and is incorporated into RNA in its place. Furthermore, 5-fluorouracil inhibits a key enzyme of pyrimidine biosynthesis: thymidilate synthase. In addition, the active ingredient provided may be the cytotoxin modified or unmodified thapsigargin. Thapsigargin is an inhibitor of the calcium-ATPase inhibitor of the endoplasmic reticulum, which greatly reduces cell growth in the capsular bag at low concentrations (100 nM) and induces cell death at higher concentrations (10-100 μM). Preferably, the thapsigargin is (meth)acrylate-modified, in order to ensure sterically unhindered covalent binding to double bonds. Thapsigargin can be correspondingly derivatized, for example, by reaction with (meth)acrylic anhydride. The term “(meth)acrylate” in the context of the disclosure is understood to mean both acrylates and methacrylates, and mixtures thereof. Paclitaxel is a spindle poison and, through binding to β-tubulin, inhibits the degradation of spindle fibers that are formed from microtubuli. This blocks mitotic cell division in the G2 phase and M phase, such that there is no cell proliferation. The compounds mentioned and their isomers and/or derivatives may thus, individually and in any combination, especially be used for inhibition of the proliferation, migration and differentiation of cells that occur in the human or animal eye. Alternatively or additionally, one or more growth factors may be provided in order to promote a wound healing reaction. The growth factor may, for example, be or include TGF-β (TGF-β1, TGF-β2, TGF-β3). In addition, active ingredient(s) provided may be one or more angiogenesis inhibitors. The angiogenesis inhibitor may be selected from a group including VEGF inhibitors, VEGFR inhibitors, antibodies, Fab fragments, single-chain variable fragments (scFv), multivalent antibody fragments (scFv multimers), peptide aptamers and peptides. VEGF inhibitors are a group of structurally different medicaments that bind to the VEGF growth factor and hence inhibit angiogenesis. VEGFR inhibitors, by contrast, are medicaments that bind to the VEGF receptor (VEGFR). These may be small molecules from the group of tyrosine kinase inhibitors (TKIs) or antibodies, especially monoclonal antibodies. In both cases, there is interruption of the intracellular signal cascade, which brings about inhibition of angiogenesis. Alternatively or additionally, Fab fragments, single-chain variable fragments (scFv), multivalent antibody fragments (scFv multimers), peptide aptamers and peptides may be provided, which prevent or at least significantly slow angiogenesis. As a result, it is possible to make a selection optimized to the respective clinical condition. Antibody fragments offer the advantage of a high binding affinity/avidity and specificity for a broad spectrum of biological target structures and haptens. Single-chain fragments can moreover be crosslinked or expressed as diabodies (60 kDa), triabodies (90 kDa), tetrabodies (120 kDa), et cetera, with different linker lengths between V domains being possible. Moreover, a particular advantage is that molecules of 60-120 kDa increase the penetration of cells and have faster clearance rates than corresponding Igs (150 kDa). In addition, it may be the case that the antibody is a diabody, triabody, tetrabody, pentabody, hexabody, heptabody or octabody. In other words, the antibody is monospecific, bispecific, trispecific, tetraspecific, pentaspecific, hexaspecific, heptaspecific, octaspecific, nonaspecific or multispecific. This allows the crosslinking of two, three, four, five, six, seven or eight target structures or target proteins, it being possible for scFv multimers to be matched particularly precisely and individually to the possibly patient-specific spatial arrangement of the target epitopes, in order to prevent angiogenesis. The increased binding valency of scFv multimers leads to particularly high avidity. The at least one angiogenesis inhibitor may especially be selected from a group including bevacizumab, brolucizumab, ranibizumab, ramucirumab, aflibercept, pegaptanib, thalidomide, axitinib, lenvatinib, lucitanib, motesanib, pazopanib, regorafenib, sorafenib, sunitinib, tivozanib, vatalanib and biosimilars thereof. In this way, it is possible to inhibit tyrosine kinases in particular, and some of the compounds mentioned, being multikinase inhibitors, are capable of inhibiting multiple protein kinases of various classes and hence of having improved efficacy.

Further benefits arise in that the active ingredient is bonded covalently to the tissue adhesive, especially via a (meth)acrylate group, and/or in that the active ingredient and the tissue adhesive take the form of an interpenetrating network. Covalent binding, optionally with use of a (meth)acrylate or a spacer, allows particularly simple and flexible immobilization of the active ingredient on the tissue adhesive, without occurrence of steric hindrance. In addition, the (meth)acrylate or the spacer, depending on the respective configuration form, can provide the necessary functional groups in order to enable the covalent binding of the active ingredient to the tissue adhesive. In some embodiments, this can also be achieved, for example, by graft polymerization. In general, other immobilization techniques such as interpenetrating or semi-/pseudo-interpenetrating networks and the like, for example, may also be provided.

In a further configuration of the disclosure, the tissue adhesive in the uncured state takes the form of a hydrogel. Alternatively or additionally, the tissue adhesive can be cured by at least one mechanism from the group of anaerobically curing, UV light-curing, anionically curing, activator-curing, moisture-curing and thermally curing. In this way, it is possible to optimize the mode and initiation of curing to the respective end use. Preference is given to UV-initiated curing.

Further benefits arise in that the tissue adhesive includes a MeTro (methacrylated recombinant tropoelastin) prepolymer and/or a GeIMA (methacrylated gelatin)/HA-NB (N-(2-aminoethyl)-4-(4-(hydroxymethyl)-2-methoxy-5-nitrosophenoxy)butanamide)-containing polymer. This achieves a highly elastic, strongly adhesive and biocompatible tissue adhesive having good adhesion to the soft tissue of the capsular bag. The preparation of a suitable MeTro prepolymer is common knowledge, for example, from Annabi et al. (“Engineering a highly elastic human protein—based sealant for surgical applications”, Sci. Transl. Med. 9, eaai7466 (2017)). MeTro prepolymers may be synthesized using recombinant human tropoelastin and methacrylic anhydride. For example, MeTro prepolymers may be synthesized with a level of methacryloyl substitution of 54% (low), 76% (moderate) and 82% (high), using 8%, 15% and 20% (v/v) methacrylic anhydride respectively. In principle, different proportions by weight or volume are alternatively possible. The MeTro hydrogels formed can then be cured by photocrosslinking with UV light (6.9 mW/cm2; 360 to 480 nm) with different exposure times of 30 to 180 s. The photoinitiator used may, for example, be [2-hydroxy-1-(4-(hydroxyethoxy)phenyl)-2-methyl-1-propanone (Irgacure 2959); 0.5%, w/v]. Alternatively or additionally, the tissue adhesive may include or consist of a GeIMA (methacrylated gelatin)/NB (N-(2-aminoethyl)-4-(4-(hydroxymethyl)-2-methoxy-5-nitrosophenoxy)butanamide)-containing polymer. This is a photoreactive polymer that simulates the composition of the extracellular matrix (ECM). This matrix hydrogel, likewise based on biomacromolecules, can be cured rapidly after UV light irradiation in order to bond the implant to the capsular bag. This polymer may additionally be bound via up to 0.1% or more of a polymerization initiator, for example lithium phenyl-2,4,6-trimethylbenzoylphosphinate (LAP), to a hydrophilic polymer which is preferably selected from a group including alginic acid, carboxymethylcellulose, chitosan, dextran, dextran sulfate, pentosan polysulfate, carrageenan, pectin, pectin derivatives, cellulose, cellulose derivatives, glucosaminoglycans, especially hyaluronic acid, dermatan sulfate, chondroitin sulfate, dermatan sulfate, keratan sulfate, heparan sulfate, heparin, heparan sulfate, hyaluronan, agarose, starch, methylcellulose, polymannuronic acid, polyguluronic acid, polyglucuronic acid, amylose, amylopectin, callose, polygalactomannan, xanthan, poly(ethylene oxide), poly(ethylene glycol), collagen, gelatin, fibrin, fibrinogen, fibronectin, vitronectin, poly(ethylene oxide), poly(acrylic acid), poly(methacrylic acid), poly(acrylamide), polyvinylpyrrolidone, poly(amino acids); poly(amines), poly(imines), a mixture thereof and/or copolymers thereof and/or pharmacologically acceptable salts thereof. For example, the hydrophilic polymer may be hyaluronic acid (HA). A suitable tissue adhesive and production thereof are known, for example, from Hong, Y., Zhou, F., Hua, Y. et al. (A strongly adhesive hemostatic hydrogel for the repair of arterial and heart bleeds. Nat Commun 10, 2060 (2019)) (cf. pages 7-9, Methods). For example, the tissue adhesive may include 1%-10%, especially 5%, methacrylated gelatin (GeIMA) and 0.5%-3%, especially 1.25%, N-(2-am inoethyl)-4-(4-(hydroxymethyl)-2-methoxy nitrosophenoxy)butanamide (NB), where the NB is bound to HA via LAP (HA-NB). In general, percent figures in the context of the present disclosure should be considered to be percent by mass, unless stated otherwise.

A second aspect of the disclosure relates to an implantation system that can reduce the risk of PCO and fibrosis in that it includes an ophthalmological implant for implantation in a human or animal eye and a tissue adhesive via which the ophthalmological implant is at least partly cohesively bondable to the patient's eye tissue. With the aid of the implantation system of the disclosure, the ophthalmological implant can be immobilized and fixedly bonded in the eye tissue, for example in the capsular bag, without damaging the tissue. It is thus possible to reliably prevent problems that commonly occur in corresponding methods of treatment, such as misalignment, tilting, rotation or detachment of the implant. Furthermore, in the case of a cataract operation, such a capsular bag-based implantation used to be associated with posterior capsule opacification (PCO) and fibrosis, which can especially be caused by residual lens epithelial cells in the equatorial region of the capsular bag. The possibility of cohesive bonding of the implant to the capsular bag with the aid of the tissue adhesive can, by contrast, reliably prevent undermining of the implant with lens epithelial cells and the like. Any remaining lens epithelial cells of the capsular bag prepared for implantation can thus no longer cause PCO and fibrosis that can impair the sight and functionality of an implant. In particular embodiments, the implant has a main body with at least one tactile section and at least one optical section, wherein the tissue adhesive may be disposed solely on the tactile section, solely on the optical section or on both sections. The tissue adhesive may in principle be present separately from the ophthalmological implant, for example in a separate package, or may already have been applied to at least a part of the implant. Further features and benefits thereof can be inferred from the description of the above aspect of the disclosure.

In a configuration of the disclosure, the tissue adhesive is designed according to the first aspect of the disclosure and/or the ophthalmological implant is an intraocular lens (IOL), especially an accommodating IOL, or an artificial capsular bag. Without the implantation of an IOL in the empty capsular bag, the risk of an aftercataract is actually increased, since unhindered cell migration to the rear or posterior surface of the capsular bag is possible in this case.

Further benefits arise in that the ophthalmological implant includes, on the outside thereof, free amino groups via which the ophthalmological implant is cohesively bondable to the capsular bag via the tissue adhesive. In other words, the tissue adhesive and the implant are matched to one another such that the tissue adhesive having free amino groups can react at the surface of the implant and form covalent bonds in order to achieve a high bonding force. For this purpose, the implant may consist at least superficially of a corresponding polymer having free amino groups. Alternatively or additionally, the implant may have a coating that provides free amino groups, for example a polyimine coating.

### BRIEF DESCRIPTION OF DRAWINGS

The invention will now be described with reference to the drawings wherein:

FIG. 1 shows a reaction for formation of methacrylated gelatin (GeIMA);

FIG. 2 shows a crosslinking reaction of GeIMA and modified hyaluronic acid (HA-NB) to produce a first network;

FIG. 3 shows a second network produced by crosslinking of the first network;

FIG. 4 shows a coupling reaction of thapsigargin to GeIMA;

FIG. 5 shows a coupling reaction of a (meth)acrylate-modified thapsigargin derivative to GeIMA; and,

FIG. 6 shows a basic diagram of an ophthalmological implantation system of the disclosure.

### DETAILED DESCRIPTION

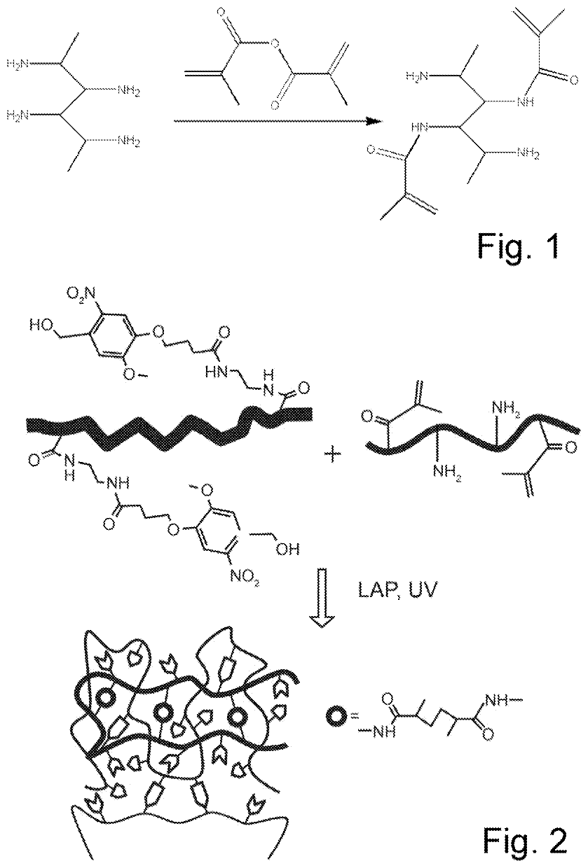

Hong, Y., Zhou, F., Hua, Y. et al. (A strongly adhesive hemostatic hydrogel for the repair of arterial and heart bleeds. Nat Commun 10, 2060 (2019)) discloses a hydrogel tissue adhesive which is similar to the composition of the extracellular matrix of biological connective tissue and is suitable for use in a method of treatment in which an eye lens of a human or animal patient is replaced by an ophthalmological implant and the ophthalmological implant is cohesively bonded to a capsular bag of the patient via the tissue adhesive. The method of treatment may, for example, be a cataract operation. This tissue adhesive forms a hydrogel and consists of about 5% methacrylated gelatin (GeIMA) and about 1.25% N-(2-aminoethyl)-4-(4-(hydroxymethyl) methoxy-5-nitrosophenoxy)butanamide (NB), bonded to the glycosaminoglycan hyaluronic acid (HA) (HA-NB). FIG. 1 shows a schematic of a reaction for formation of GeIMA in which gelatin is mixed with methacrylic anhydride and optionally kept at 50° C. in DPBS (Dulbecco's phosphate-buffered saline) while stirring for 48 hours. NB is in turn bound to HA and crosslinked with GeIMA in order to form a first GelMA/HA-NB network. The crosslinking reaction is started by UV photoactivation of the polymerization initiator lithium phenyl-2,4,6-trimethylbenzoylphosphinate (LAP) (0.1%). The crosslinking reaction of GeIMA and modified hyaluronic acid (HA-NB) to produce a first network is shown schematically in FIG. 2.

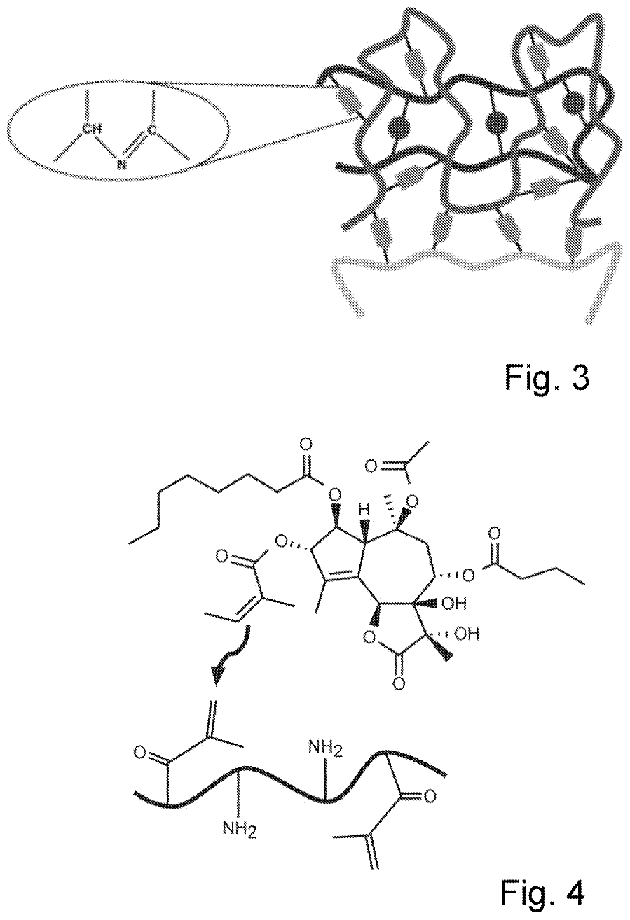

The UV irradiation converts hydroxymethyl groups of NA to keto groups, which react with free amino groups of GeIMA to give Schiff bases, and in so doing form a second network. The resulting second network is shown schematically in FIG. 3. The resulting tissue adhesive binds strongly to moist biological tissue surfaces after UV photoactivation of the LAP.

The cytotoxin thapsigargin which is shown in FIG. 4 is an inhibitor of the calcium-ATPase inhibitor of the endoplasmic reticulum, which greatly reduces cell growth in the capsular bag at low concentrations (100 nM) and induces cell death at higher concentrations (10-100 μM). It can therefore be used in principle for prevention of PCO and fibrosis. Free thapsigargin which is released into the aqueous humor in the anterior chamber, but can damage the epithelial cell layer of the cornea.

In order to prevent uncontrolled release of thapsigargin, according to FIG. 4, the acrylate groups of thapsigargin are bound covalently to GeIMA. For this purpose, the thapsigargin is added to the aforementioned tissue adhesive, such that the thapsigargin likewise binds covalently to the GeIMA through the employment of UV and LAP, which is used for curing the tissue adhesive.

The bound thapsigargin cannot display any toxic effect because it has to get into the cells to do so. The aqueous humor of the eye contains matrix metalloproteinases, the concentration of which rises during the cataract operation owing to an elevated TGFβ level. Matrix metalloproteinases are gelatinases that digest collagens and gelatin. In the presence of these matrix metalloproteinases, thapsigargin-containing GeIMA is degraded with time, which can achieve controlled release of thapsigargin in an active ingredient release system. As a result of the incorporation into the tissue adhesive, a small amount of thapsigargin is released over an adjustable period of time only in the immediate proximity of the tissue adhesive and hence in the immediate proximity of PCO- and fibrosis-causing cells, without damaging other tissue.

It should be emphasized that it is also possible to provide other active pharmacological ingredients, which are incorporated in these or other suitable tissue adhesives, are covalently bound thereto, or form a composition therewith in some other way. The composition may be produced before, during and/or after the ultimate mixing of the tissue adhesive components and the curing by LAP/UV. The active ingredient(s) need not necessarily bind to the acryloyl groups of the tissue adhesive; alternatively or additionally, a bond to the free amino groups of the GeIMA may also be provided.

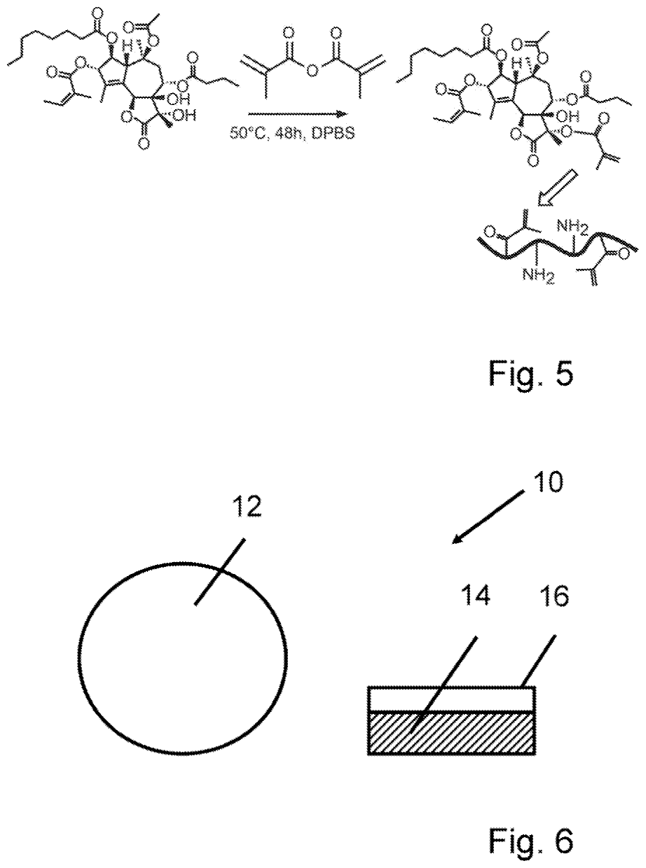

FIG. 5 shows a schematic coupling of modified thapsigargin to GeIMA. The thapsigargin in the present case is bound covalently by an acrylate group to GeIMA or to the tissue adhesive 14. This has the benefit of lower steric hindrance and a correspondingly simpler reaction regime with higher and faster conversion. For this purpose, the thapsigargin is first derivatized with methacrylic anhydride at about 50° C. for 48 hours in phosphate-buffered salt solution (DPBS—Dulbecco's Phosphate-Buffered Saline). The methacrylate group binds here to free OH groups of thapsigargin. Subsequently, the derivatized thapsigargin binds covalently via that methacrylate group to a corresponding methacrylate group in the modified gelatin (GeIMA). After the eye operation and the implantation of the implant 12, the already described degradation by matrix metalloproteinases (MMP) results in slow release of the thapsigargin in the operation region.

FIG. 6 shows a basic diagram of an ophthalmological implantation system 10 of the disclosure. The implantation system 10 includes an ophthalmological implant 12, via which the lens of a patient's eye is replaceable. The implant 12 may take the form, for example, of an accommodating intraocular lens. Alternatively, the implant 12 may also be a different type of implant, for example a non-accommodating IOL, an (optionally accommodating) IOL with one or more tactile portions or an artificial capsular bag (not shown). A suitable artificial capsular bag in which an IOL is implantable in turn is known, for example, from U.S. Pat. No. 8,900,300 B1. In addition, the implantation system 10 includes a tissue adhesive 14, via which the ophthalmological implant 12, after implantation thereof, is cohesively bondable to a patient's capsular bag. The tissue adhesive 14 may take the form as described above and be stored in a suitable pack 16 until use thereof. Alternatively, the tissue adhesive 14 may already have been applied to the implant 12. In that case, the implant 12 and the tissue adhesive 14 are preferably stored so as to prevent premature curing of the tissue adhesive 14, that is, curing during storage or before implantation.

The parameter values specified in the documents to define process and measurement conditions for the characterization of specific properties of the subject matter of the disclosure should also be considered to be encompassed by the scope of the disclosure within the scope of variances—for example due to measurement errors, system errors, weighing errors, and the like.

It is understood that the foregoing description is that of the preferred embodiments of the invention and that various changes and modifications may be made thereto without departing from the spirit and scope of the invention as defined in the appended claims.

### LIST OF REFERENCE SYMBOLS

10 implantation system
12 implant
14 tissue adhesive
16 pack

1. A tissue adhesive comprising a hydrogel,
wherein the tissue adhesive in an uncured state and is formed as an interpenetrating network and/or a semi-interpenetrating network,
wherein the hydrogel is an active ingredient release system which comprises: (i) MeTro (methacrylated recombinant tropoelastin) prepolymer, and/or (ii) a GeIMA (methacrylated gelatin) combined with N-(2-aminoethyl)-4-(4-(hydroxymethyl)-2-methoxy-5-nitrosophenoxy)butanamide),
wherein the active ingredient promotes and/or inhibits proliferation, migration, and/or differentiation of eye cells, and
wherein the active ingredient is one or more of 5-fluorouracil, thapsigargin, paclitaxel, growth factors angiogenesis inhibitors, and (meth)acrylates.

2. The tissue adhesive of claim 1, wherein the active ingredient is bonded covalently to the hydrogel.

3. The tissue adhesive of claim 1, further comprising a biopolymer.

4. An ophthalmological implantation system comprising an ophthalmological implant and the tissue adhesive of claim 1, wherein the ophthalmological implant is an intraocular lens or an artificial capsular bag.

5. The tissue adhesive of claim 1, wherein the active ingredient is transforming growth factor β (TGFβ).

6. The tissue adhesive of claim 3, wherein the biopolymer comprises hyaluronic acid.

7. The tissue adhesive of claim 1, further comprising one or more hydrophilic polymers selected from alginic acid, carboxymethylcellulose, chitosan, dextran, dextran sulfate, pentosan polysulfate, carrageenan, pectin, pectin derivatives, cellulose, cellulose derivatives, glucosaminoglycans, especially hyaluronic acid, dermatan sulfate, chondroitin sulfate, dermatan sulfate, keratan sulfate, heparan sulfate, heparin, heparan sulfate, hyaluronan, agarose, starch, methylcellulose, polymannuronic acid, polyguluronic acid, polyglucuronic acid, amylose, amylopectin, callose, polygalactomannan, xanthan, poly(ethylene oxide), poly(ethylene glycol), collagen, gelatin, fibrin, fibrinogen, fibronectin, vitronectin, poly(ethylene oxide), poly(acrylic acid), poly(methacrylic acid), poly(acrylamide), polyvinylpyrrolidone, poly(amino acids); poly(amines), and poly(imines).

8. A method of implanting an ocular implant into an eye of a subject in need thereof, which comprises:
providing an implant comprising a surface,
providing a tissue adhesive,
applying the tissue adhesive to a surface of the implant, contacting the eye of the subject with the implant surface, thereby cohesively bonding the implant to the eye of the subject,
wherein the tissue adhesive comprises a hydrogel, which is in an uncured state and is formed as an interpenetrating network and/or a semi-interpenetrating network,
wherein the hydrogel is an active ingredient release system which comprises: (i) MeTro (methacrylated recombinant tropoelastin) prepolymer, and/or (ii) a GeIMA (methacrylated gelatin) combined with N-(2-aminoethyl)-4-(4-(hydroxymethyl)-2-methoxy-5-nitrosophenoxy)butanamide),
wherein the active ingredient promotes and/or inhibits proliferation, migration, and/or differentiation of eye cells, and
wherein the active ingredient is one or more of 5-fluorouracil, thapsigargin, paclitaxel, growth factors angiogenesis inhibitors, and (meth)acrylate.

9. The method of claim 8, further comprising crosslinking the tissue adhesive.

10. The method of claim 8, wherein crosslinking is achieved by anaerobic curing, UV light-curing, anionical curing, activator-curing, moisture-curing, and/or thermal curing.

11. A tissue adhesive for use in attaching an ophthalmological implant to a human or animal patient,
wherein the ophthalmological implant is at least partly cohesively bonded to the patient's eye tissue via the tissue adhesive,
wherein the tissue adhesive in an uncured state is formed as an interpenetrating network and/or a semi-interpenetrating network,
wherein the adhesive is provided as a composition with an active ingredient release system which is designed to release at least one active pharmacological ingredient when implanted,
wherein the at least one active ingredient is designed to promote and/or inhibit at least one aspect from the group consisting of: proliferation, migration and differentiation of cells that occur in the human or the animal eye, and
wherein the at least one active ingredient is selected from the group consisting of: 5-fluorouracil, thapsigargin, paclitaxel, growth factors, angiogenesis inhibitors, (meth)acrylate-modified compounds, isomers, and mixtures thereof.

12. The tissue adhesive of claim 11, wherein the adhesive is provided as a composition with an active ingredient release system designed, in the implanted state of the tissue adhesive, to release at least one active pharmacological ingredient in a controlled manner, and/or in that the tissue adhesive is provided as a composition with an active pharmacological ingredient immobilized on the tissue adhesive.

13. The tissue adhesive of claim 11, wherein the active ingredient is bonded covalently to the tissue adhesive via a (meth)acrylate group, and/or wherein the active ingredient and the tissue adhesive are in the form of the interpenetrating network.

14. The tissue adhesive of claim 11, wherein in the uncured state the adhesive is in the form of a hydrogel, the interpenetrating network, and/or the semi-interpenetrating network, and/or wherein the adhesive can be cured by at least one mechanism selected from the group consisting of: anaerobic curing, UV light-curing, anionic curing, activator-curing, moisture-curing, and thermally curing.

15. The tissue adhesive of claim 11, wherein the adhesive comprises a MeTro (methacrylated recombinant tropoelastin) prepolymer and/or a GeIMA (methacrylated gelatin)/NB (N-(2-aminoethyl)-4-(4-(hydroxymethyl)-2-methoxy-5-nitrosophenoxy)butanamide)-containing polymer, and/or
wherein the adhesive comprises the adhesive comprises a MeTro (methacrylated recombinant tropoelastin) prepolymer and/or the GeIMA (methacrylated gelatin)/NB (N-(2-aminoethyl)-4-(4-(hydroxymethyl)-2-methoxy-5-nitrosophenoxy)butanamide)-containing polymer, combined with a biopolymer, wherein the biopolymer is hyaluronic acid (HA).

16. The tissue adhesive of claim 11, wherein the at least one active ingredient is TGFβ.

17. The tissue adhesive of claim 11, wherein the at least one active ingredient is bonded covalently to the tissue adhesive via a (meth)acrylate group.

18. An ophthalmological implantation system comprising an ophthalmological implant for implantation in a human or animal eye and a tissue adhesive via which the ophthalmological implant is at least partly cohesively bondable to the patient's eye tissue, wherein the tissue adhesive is as claimed in claim 11.

19. The ophthalmological implantation system of claim 18, wherein the ophthalmological implant is an intraocular lens or an artificial capsular bag.

20. The ophthalmological implantation system of claim 18, wherein the ophthalmological implant comprises, on an outside thereof, free amino groups via which the ophthalmological implant is cohesively bondable to the eye tissue via the tissue adhesive.
